(19) United States
(12) Patent Application Publication
(10) Pub. No.: US 2025/0027044 A1
(43) Pub. Date: Jan. 23, 2025

(54) Creation of Inducible Pluripotent Stem Cell Derived T Regulatory Cells by In Vitro Recapitulation ofThymic Development

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/775,133

(22) Filed: Jul. 17, 2024

Related U.S. Application Data
(60) Provisional application No. 63/514,240, filed on Jul. 18, 2023.

(51) Int. Cl. C12N 5/0783

(52) U.S. Cl. CPC C12N 5/0637; C12N 2501/24; C12N 2501/25; C12N 2502/078; C12N 2506/45

(57) ABSTRACT

Compositions of matter and means useful for treatment of autoimmunity and/or transplant rejection through generation of T regulatory cells in a manner that recapitulates thymic development. Methods of seeding thymic medullary epithelial cells with pluripotent stem cells such as inducible pluripotent stem cells, followed by exposure to conditions allowing for selective growth and expansion of T regulatory cells. T regulatory cells can be scaled for clinical utilization through the use of three-dimensional adhesive growth matrices as well as addition of FoxP3 inducing cytokines such as interleukin-2.

### CROSS-REFERENCE TO RELATED APPLICATIONS

The present application claims benefit of U.S. Provisional Patent Application Ser. No. 63/514,240, filed on Jul. 18, 2023, entitled CREATION OF INDUCIBLE PLURIPOTENT STEM CELL DERIVED T REGULATORY CELLS BY IN VITRO RECAPITULATION OFTHYMIC DEVELOPMENT, the contents of which are incorporated herein by reference in its entirety.

### FIELD OF THE INVENTION

The adaptive immune response is coordinated by cells capable of immunological memory, as well as clonotypic expansion, such as T cells and B cells. It is accepted that a fundamental function of adaptive immunity is the capacity to respond to a previously encountered antigen efficiently and more rapidly. From the T cell compartment it is recognized that these cells are generally divided into T helper cells, which are CD4 expressing T cells, and T cytotoxic cells which are CD8 expressing T cells. CD4+ T cells comprise a mixed population of T cells which are of fundamental importance in both the generation of immune responses and the suppression of autoimmune diseases. A subpopulation of CD4+ T cells expresses the transcription factor forkhead box P3 (Foxp3). This subpopulation, loosely defined as regulatory T cells or Tregs, plays a pivotal role in maintaining self-tolerance.

These T regulatory cells (Tregs) are functionally defined as T cells that inhibit the immune response by influencing the activity of another cell. These cells comprise a small population of thymus derived CD4+ T cells. Despite there being a small population, Tregs have a large regulatory effect on the immune system.

Functionally, Tregs inhibit immunity through various mechanisms. One of the most common is blockade of T cell proliferation and cytokine secretion by expression of TGF-beta on their surface. This cytokine is capable of abrogating intracellular signaling of the T cell receptor. Another mechanism is suppression of dendritic cell maturation. While dendritic cells are the only cell capable of activating naïve T cells, immature dendritic cells induce generation of anergic T cells, which are dysfunctional. Other mechanisms have been proposed including direct killing of activity T cells by a perforin-based mechanism.

At a clinical level, is accepted that Tregs suppress the development of inflammatory and autoimmune disorders. For example, lack of Tregs causes autoimmune gastritis, thyroiditis, insulin-dependent diabetes mellitus (IDDM), inflammatory bowel disorders (IBD), experimental autoimmune encephalomyelitis (EAE), food allergies, and graft rejection.

Because of the ability of Tregs to suppress autoimmunity, pregnancy loss, and transplant rejection, there has been increasing clinical interest in the application of Tregs as a part of “anti-immunotherapy” to actively block autoimmune diseases, allergies and transplantation-related complications, such as graft rejection or graft-versus-host disease (GvHD).

Unfortunately, in vitro generation of Tregs in sufficient quantities and with adequate potency for clinical use has not been achieved. This is in part because current protocols for Treg expansion do not generate clinically relevant numbers of Treg cells. Conversely, protocols used to “hyperaccelerate” Treg generation often lead to loss of suppressive activity.

The current invention recapitulates normal developmental process of Treg cells in vitro in order to create sufficient number of these cells which maintain potency for clinical applications. The cells generated by the invention may be utilized in an autologous, allogeneic, or xenogeneic manner.

### BACKGROUND OF THE INVENTION

Immunological dogma states that many autoimmune and inflammatory diseases involve autoreactive T-cells. For example, Multiple Sclerosis (MS), Rheumatoid Arthritis (RA), Systemic Lupus Erythromatosus (SLE), and Type 1 Diabetes are all autoimmune conditions. Current treatments for autoimmune and inflammatory diseases generally suppress the immune system. For example, one treatment includes transplantation of bone marrow along with administration of cytostatics and immunosuppressive drugs. Autologous hematopoietic stem cell transplantation can have lasting beneficial effects for some patients, but the procedure requires aggressive myelo-ablative conditioning which is associated with substantial toxicity and risk. Although several disease-modifying treatments (DMTs) have been approved to reduce the frequency of clinical relapses, most patients continue to clinically deteriorate under current therapy schedules. Neither DMTs nor stem cell transplantation can mediate specific suppression of the immunopathology of autoimmune and inflammatory diseases. Currently, effective treatments for autoimmune and inflammatory diseases do not exist. Treatment is focused on merely reducing its symptoms, usually by general suppression of the immune system. There is a need for a therapy which specifically targets local immune responses associated with onset and progression of disease.

Although T regulatory cell therapies have demonstrated clinical signals, these therapies need optimization.

### SUMMARY OF THE INVENTION

Preferred methods include embodiments of generating T regulatory cells comprising the steps of: a) obtaining a pluripotent stem cell population; b) exposing said pluripotent stem cell population to a population of thymic medullary epithelial cells; c) culturing the populations of “a” and “b” together; d) providing conditions for differentiation of said pluripotent stem cells into T regulatory cells and e) further providing conditions for expansion of said T regulatory cells.

Preferred methods include embodiments wherein said pluripotent stem cell is an inducible pluripotent stem cell.

Preferred embodiments include methods wherein said pluripotent stem cell is substitute with a lymphoid progenitor cell.

Preferred embodiments include methods wherein said lymphoid progenitor cell expresses FoxP3.

Preferred embodiments include methods wherein said lymphoid progenitor cell expresses CD25.

Preferred embodiments include methods wherein said lymphoid progenitor cell expresses reduced CD127 as compared to T cells.

Preferred embodiments include methods wherein said T cell is a CD4 T cell.

Preferred embodiments include methods wherein said pluripotent stem cell is substituted with a T cell progenitor.

Preferred embodiments include methods wherein said pluripotent stem cell is substituted with a peripheral blood T cell.

Preferred embodiments include methods wherein said pluripotent stem cell is substituted with a CD4 expressing peripheral blood T cell.

Preferred embodiments include methods wherein said pluripotent stem cell is substituted with a CD25 expressing peripheral blood T cell.

Preferred embodiments include methods wherein said pluripotent stem cell is substituted with a CTLA4 expressing peripheral blood T cell.

Preferred embodiments include methods wherein said pluripotent stem cell is substituted with an AIRE expressing peripheral blood T cell.

Preferred embodiments include methods wherein said pluripotent stem cell is an embryonic stem cell.

Preferred embodiments include methods wherein said pluripotent stem cell expresses hTERT.

Preferred embodiments include methods wherein said pluripotent stem cell expresses SSEA4.

Preferred embodiments include methods wherein said pluripotent stem cell expresses NANOG.

Preferred embodiments include methods wherein said pluripotent stem cell expresses PIM-1.

Preferred embodiments include methods wherein said pluripotent stem cell expresses SOX-2.

Preferred embodiments include methods wherein said pluripotent stem cell expresses KLF4.

Preferred embodiments include methods wherein said pluripotent stem cell expresses c-met.

Preferred embodiments include methods wherein said pluripotent stem cell expresses CD123.

Preferred embodiments include methods wherein said pluripotent stem cell expresses leukemia inhibitory factor receptor.

Preferred embodiments include methods wherein said T regulatory cell is capable of suppressing proliferation of peripheral blood mononuclear cells stimulated with a mitogenic lectin.

Preferred embodiments include methods wherein said T regulatory cell is capable of suppressing proliferation of peripheral blood mononuclear cells stimulated with a mitogenic cytokine.

Preferred embodiments include methods wherein said T regulatory cell is capable of suppressing proliferation of peripheral blood mononuclear cells stimulated by ligation of CD3.

Preferred embodiments include methods wherein said T regulatory cell is capable of suppressing proliferation of peripheral blood mononuclear cells stimulated by ligation of CD3 and CD28.

Preferred embodiments include methods wherein said T regulatory cell is capable of suppressing proliferation of peripheral blood mononuclear cells stimulated by ligation of CD3, CD28 and ICAM-1.

Preferred embodiments include methods wherein said mitogenic cytokine is interleukin-2.

Preferred embodiments include methods wherein said mitogenic cytokine is interleukin-3.

Preferred embodiments include methods wherein said mitogenic cytokine is interleukin-7.

Preferred embodiments include methods wherein said mitogenic cytokine is interleukin-12.

Preferred embodiments include methods wherein said mitogenic cytokine is interleukin-15.

Preferred embodiments include methods wherein said mitogenic cytokine is interleukin-17.

Preferred embodiments include methods wherein said mitogenic cytokine is interleukin-18.

Preferred embodiments include methods wherein said mitogenic cytokine is interleukin-33.

Preferred embodiments include methods wherein said mitogenic cytokine is interferon alpha.

Preferred embodiments include methods wherein said mitogenic cytokine is interferon gamma.

Preferred embodiments include methods wherein said mitogenic cytokine is granulin.

Preferred embodiments include methods wherein said mitogenic cytokine is soluble vimentin.

Preferred embodiments include methods wherein said T regulatory cell is capable of suppressing production of interferon gamma by peripheral blood mononuclear cells stimulated with a mitogenic lectin.

Preferred embodiments include methods wherein said T regulatory cell is capable of suppressing production of interferon gamma by peripheral blood mononuclear cells stimulated with a mitogenic cytokine.

Preferred embodiments include methods wherein said T regulatory cell is capable of suppressing production of interferon gamma by peripheral blood mononuclear cells stimulated by ligation of CD3.

Preferred embodiments include methods wherein said T regulatory cell is capable of suppressing production of interferon gamma by peripheral blood mononuclear cells stimulated by ligation of CD3 and CD28.

Preferred embodiments include methods wherein said T regulatory cell is capable of suppressing production of interferon gamma by peripheral blood mononuclear cells stimulated by ligation of CD3, CD28 and ICAM-1.

Preferred embodiments include methods wherein said mitogenic cytokine is interleukin-2.

Preferred embodiments include methods wherein said mitogenic cytokine is interleukin-3.

Preferred embodiments include methods wherein said mitogenic cytokine is interleukin-7.

Preferred embodiments include methods wherein said mitogenic cytokine is interleukin-12.

Preferred embodiments include methods wherein said mitogenic cytokine is interleukin-15.

Preferred embodiments include methods wherein said mitogenic cytokine is interleukin-17.

Preferred embodiments include methods wherein said mitogenic cytokine is interleukin-18.

Preferred embodiments include methods wherein said mitogenic cytokine is interleukin-33.

Preferred embodiments include methods wherein said mitogenic cytokine is interferon alpha.

Preferred embodiments include methods wherein said mitogenic cytokine is interferon gamma.

Preferred embodiments include methods wherein said mitogenic cytokine is granulin.

Preferred embodiments include methods wherein said mitogenic cytokine is soluble vimentin.

Preferred embodiments include methods wherein said T regulatory cell is capable of suppressing dendritic cell maturation.

Preferred embodiments include methods wherein said T regulatory cell suppresses said dendritic cell maturation in a SMAD dependent manner.

Preferred embodiments include methods wherein said T regulatory cell suppresses said dendritic cell maturation in a TGF-beta dependent manner.

Preferred embodiments include methods wherein said T regulatory cell suppresses said dendritic cell maturation in a GITR dependent manner.

Preferred embodiments include methods wherein said immature dendritic cell possesses enhanced migratory activity as compared to a mature dendritic cell.

Preferred embodiments include methods wherein said migratory activity is chemotaxis towards a chemotactic gradient.

Preferred embodiments include methods wherein said chemotactic gradient is stimulated by inflammation.

Preferred embodiments include methods wherein said chemotactic gradient is stimulated by hypoxia.

Preferred embodiments include methods wherein said chemotactic gradient is stimulated by NF-kappa B activation.

Preferred embodiments include methods wherein said chemotactic gradient is stimulated by hypoxia inducible factor activation (HIF).

Preferred embodiments include methods wherein said chemotactic gradient is stimulated by tissue factor release.

Preferred embodiments include methods wherein said chemotactic gradient is stimulated by low molecular weight hyaluronic acid fragments.

Preferred embodiments include methods wherein said chemotactic gradient is stimulated by activation of one or more matrix metalloproteases.

Preferred embodiments include methods wherein said chemotactic gradient is stimulated by inhibition of one or more tissue inhibitors of metalloproteinases (TIMPS).

Preferred embodiments include methods wherein said chemotactic gradient is stimulated by release of endogenous tissue derived toll like receptor agonists.

Preferred embodiments include methods wherein said chemotactic gradient is stimulated by HMGB1.

Preferred embodiments include methods wherein said chemotactic gradient is stimulated by neutrophil extracellular traps.

Preferred embodiments include methods wherein said chemotactic gradient is stimulated by free histones.

Preferred embodiments include methods wherein said chemotactic gradient is stimulated by hsp27.

Preferred embodiments include methods wherein said chemotactic gradient is stimulated by gp96.

Preferred embodiments include methods wherein said chemotactic gradient is stimulated by calreticulin.

Preferred embodiments include methods wherein said chemotactic gradient is stimulated by extracellular actin.

Preferred embodiments include methods wherein said chemotactic gradient is stimulated by extracellular tau.

Preferred embodiments include methods wherein said chemotactic gradient is stimulated by synuclein.

Preferred embodiments include methods wherein said chemotactic gradient is stimulated by peroxidized lipids.

Preferred embodiments include methods wherein said chemotactic gradient is stimulated by ozonized lipids.

Preferred embodiments include methods wherein said immature dendritic cell is capable of stimulating generation of anergic T cells in an antigen-dependent manner.

Preferred embodiments include methods wherein said immature dendritic cell is capable of stimulating generation of anergic T cells in an antigen-independent manner.

Preferred embodiments include methods wherein said anergic T cell does not proliferate in response to mitogenic signals.

Preferred embodiments include methods wherein said anergic T cell does not produce cytokines in response to mitogenic signals.

Preferred embodiments include methods wherein said anergic T cell does not produce interferon gamma in response to mitogenic signals.

Preferred embodiments include methods wherein said anergic T cell does not produce interleukin-2 in response to mitogenic signals.

Preferred embodiments include methods wherein said anergic T cell does not generate cytotoxic effector molecules in response to mitogenic signals.

Preferred embodiments include methods wherein said cytotoxic effector molecule is granzyme B.

Preferred embodiments include methods wherein said cytotoxic effector molecule is perforin.

Preferred embodiments include methods wherein said cytotoxic effector molecule is TRAIL.

Preferred embodiments include methods wherein said cytotoxic effector molecule is RANK-ligand.

Preferred embodiments include methods wherein said cytotoxic effector molecule is Fas ligand.

Preferred embodiments include methods wherein said anergic T cells produce interleukin-10 in response to mitogenic signals.

Preferred embodiments include methods wherein said anergic T cells produce interleukin-4 in response to mitogenic signals.

Preferred embodiments include methods wherein said anergic T cells produce interleukin-13 in response to mitogenic signals.

Preferred embodiments include methods wherein said anergic T cells produce interleukin-20 in response to mitogenic signals.

Preferred embodiments include methods wherein said anergic T cells produce VEGF in response to mitogenic signals.

Preferred embodiments include methods wherein said anergic T cells produce interleukin-35 in response to mitogenic signals.

Preferred embodiments include methods wherein dendritic cells made immature by Treg cells produce more interleukin-10 as compared to mature dendritic cells.

Preferred embodiments include methods wherein dendritic cells made immature by Treg cells produce more in TGF-beta as compared to mature dendritic cells.

Preferred embodiments include methods wherein dendritic cells made immature by Treg cells produce more interleukin-35 as compared to mature dendritic cells.

Preferred embodiments include methods wherein said dendritic cells made immature by Treg cells produce more interleukin 1 receptor antagonist as compared to mature dendritic cells.

Preferred embodiments include methods wherein said dendritic cells made immature by Treg cells produce more endoglin as compared to mature dendritic cells.

Preferred embodiments include methods wherein said dendritic cells made immature by Treg cells produce more progesterone induced blocking factor as compared to mature dendritic cells.

Preferred embodiments include methods wherein said dendritic cells made immature by Treg cells produce more soluble HLA-G as compared to mature dendritic cells.

Preferred embodiments include methods wherein said dendritic cells made immature by Treg cells produce more hepatocyte growth factor as compared to mature dendritic cells.

Preferred embodiments include methods wherein said dendritic cells made immature by Treg cells express more PD-L1 as compared to mature dendritic cells.

Preferred embodiments include methods wherein dendritic cells made immature by Treg cells express more arginase as compared to mature dendritic cells.

Preferred embodiments include methods wherein said dendritic cells made immature by Treg cells express more indolamine 2,3 dioxygenase as compared to mature dendritic cells.

Preferred embodiments include methods wherein said dendritic cells made immature by Treg cells express less CD40 as compared to mature dendritic cells.

Preferred embodiments include methods wherein said dendritic cells made immature by Treg cells express less CD80 as compared to mature dendritic cells.

Preferred embodiments include methods wherein said dendritic cells made immature by Treg cells express less CD86 as compared to mature dendritic cells.

Preferred embodiments include methods wherein said dendritic cells made immature by Treg cells express less IL-12 as compared to mature dendritic cells.

Preferred embodiments include methods wherein said dendritic cells made immature by Treg cells express less TLR4 as compared to mature dendritic cells.

Preferred embodiments include methods wherein said dendritic cells made immature by Treg cells express less TLR3 as compared to mature dendritic cells.

Preferred embodiments include methods wherein said dendritic cells made immature by Treg cells express more TLR2 as compared to mature dendritic cells.

Preferred embodiments include methods wherein said dendritic cells made immature by Treg cells express more TLR5 as compared to mature dendritic cells.

Preferred embodiments include methods wherein said dendritic cells made immature by Treg cells stimulate generation of T regulatory cells as compared to mature dendritic cells.

Preferred embodiments include methods wherein said thymic medullary epithelial cells are isolated from living or cadaveric donors.

Preferred embodiments include methods wherein said thymic medullary epithelial cells are dissociated from donor thymus based on expression of PECAM-1.

Preferred embodiments include methods wherein said thymic medullary epithelial cells are dissociated from donor thymus based on expression of syndecan.

Preferred embodiments include methods wherein said thymic medullary epithelial cells are dissociated from donor thymus based on expression of PECAM-1 and syndecan.

Preferred embodiments include methods wherein said thymic medullary epithelial cells are dissociated from donor thymus based on expression of AIRE.

Preferred embodiments include methods wherein said thymic medullary epithelial cells are dissociated from donor thymus based on expression of proteins encoded by AIRE target genes.

Preferred embodiments include methods wherein said protein encoded by AIRE target gene is fas ligand.

Preferred embodiments include methods wherein said protein encoded by AIRE target gene is HLA-G.

Preferred embodiments include methods wherein said protein encoded by AIRE target gene is CD80.

Preferred embodiments include methods wherein said protein encoded by AIRE target gene is proinsulin.

Preferred embodiments include methods wherein said protein encoded by AIRE target gene is S100a8.

Preferred embodiments include methods wherein said protein encoded by AIRE target gene is DNA-dependent protein kinase.

Preferred embodiments include methods wherein said protein encoded by AIRE target gene is topoisomerase 2a.

Preferred embodiments include methods wherein said protein encoded by AIRE target gene is topoisomerase 1.

Preferred embodiments include methods wherein said protein encoded by AIRE target gene is CAMK2B.

Preferred embodiments include methods wherein said protein encoded by AIRE target gene is CCL3.

Preferred embodiments include methods wherein said protein encoded by AIRE target gene is CCL5.

Preferred embodiments include methods wherein said protein encoded by AIRE target gene is CEACAM-1.

Preferred embodiments include methods wherein said protein encoded by AIRE target gene is KRT14.

Preferred embodiments include methods wherein said protein encoded by AIRE target gene is KRT17.

Preferred embodiments include methods wherein said protein encoded by AIRE target gene is LEFTY2.

Preferred embodiments include methods wherein said protein encoded by AIRE target gene is OAS3.

Preferred embodiments include methods wherein thymic epithelial cells are obtained through extraction of thymic epithelial progenitor cells (TEPCs).

Preferred embodiments include methods wherein said thymic epithelial cells are derived by contacting the cells, or one or more ancestors thereof, with at least one thymic epithelial cell promoting agent, and then allowing or causing the TEPCs to differentiate.

Preferred embodiments include methods wherein at least one promoting agent inhibits differentiation of the TEPCs into cortical and/or medullary thymic epithelial cells.

Preferred embodiments include methods wherein said promoting agent is interleukin-7.

Preferred embodiments include methods wherein said promoting agent is interleukin-15.

Preferred embodiments include methods wherein said promoting agent is interleukin-12 p40 homodimer.

Preferred embodiments include methods wherein said promoting agent is interleukin-35.

Preferred embodiments include methods wherein said promoting agent is IGF-1.

Preferred embodiments include methods wherein said promoting agent is FGF-1.

Preferred embodiments include methods wherein said promoting agent is FGF-2.

Preferred embodiments include methods wherein said promoting agent is FGF-7.

Preferred embodiments include methods wherein said promoting agent is FGF-12.

Preferred embodiments include methods wherein said promoting agent is hydrocortisone.

Preferred embodiments include methods wherein said promoting agent is transferrin.

Preferred embodiments include methods wherein said promoting agent is high density lipoprotein.

Preferred embodiments include methods wherein said promoting agent is ascorbic acid.

Preferred embodiments include methods wherein said promoting agent is linolenic acid.

Preferred embodiments include methods wherein said promoting agent is thymosin beta.

Preferred embodiments include methods wherein said promoting agent is thymocyte conditioned media.

Preferred embodiments include methods wherein said promoting agent is fibroblast conditioned media.

Preferred embodiments include methods wherein said promoting agent is mesenchymal stem cell conditioned media.

Preferred embodiments include methods wherein said mesenchymal stem cell conditioned media is generated by activation of said mesenchymal stem cell.

Preferred embodiments include methods wherein said mesenchymal stem cell is umbilical tissue derived.

Preferred embodiments include methods wherein said mesenchymal stem cell is bone marrow tissue derived.

Preferred embodiments include methods wherein said mesenchymal stem cell is adipose tissue derived.

Preferred embodiments include methods wherein said mesenchymal stem cell is endometrial tissue derived.

Preferred embodiments include methods wherein said mesenchymal stem cell is fallopian tube tissue derived.

Preferred embodiments include methods wherein said mesenchymal stem cell is umbilical cord blood derived.

Preferred embodiments include methods wherein said mesenchymal stem cell is peripheral blood derived.

Preferred embodiments include methods wherein said mesenchymal stem cell is cardiosphere derived.

Preferred embodiments include methods wherein said mesenchymal stem cell is deciduous tooth derived.

Preferred embodiments include methods wherein said mesenchymal stem cell is nail cuticle derived.

Preferred embodiments include methods wherein said mesenchymal stem cell is hair follicle.

Preferred embodiments include methods wherein said mesenchymal stem cell is dermal derived.

Preferred embodiments include methods wherein said mesenchymal stem cell is activated with lipopolysaccharide.

Preferred embodiments include methods wherein said mesenchymal stem cell is activated with high mobility group box-1 protein.

Preferred embodiments include methods wherein said mesenchymal stem cell is activated with TNF-alpha.

Preferred embodiments include methods wherein said mesenchymal stem cell is activated with a member of the TNF-alpha superfamily.

Preferred embodiments include methods wherein said mesenchymal stem cell is activated with osteopontin.

Preferred embodiments include methods wherein said mesenchymal stem cell is activated with Poly IC.

Preferred embodiments include methods wherein said mesenchymal stem cell is activated with flagellin.

Preferred embodiments include methods wherein said mesenchymal stem cell is activated with low molecular weight hyaluronic acid.

Preferred embodiments include methods wherein said mesenchymal stem cell is activated with lymphotoxin.

Preferred embodiments include methods wherein said mesenchymal stem cell is activated with neutrophil extracellular traps.

Preferred embodiments include methods wherein said mesenchymal stem cell is activated with imiquimod.

Preferred embodiments include methods wherein said mesenchymal stem cell is activated with OK231.

Preferred embodiments include methods wherein said mesenchymal stem cell is activated with BCG.

Preferred embodiments include methods wherein said mesenchymal stem cell is activated with beta glucan.

Preferred embodiments include methods wherein said mesenchymal stem cell is activated with interferon gamma.

Preferred embodiments include methods wherein said mesenchymal stem cell is activated with TNF-alpha and interferon gamma.

Preferred embodiments include methods wherein said mesenchymal stem cell is activated by exposure to allogeneic T cells.

Preferred embodiments include methods wherein said mesenchymal stem cell is activated by treatment with interleukin-6.

Preferred embodiments include methods wherein said mesenchymal stem cell is activated with interleukin-1 beta.

Preferred embodiments include methods wherein said mesenchymal stem cell is activated with interleukin-17.

Preferred embodiments include methods wherein said mesenchymal stem cell is activated with interleukin-18.

Preferred embodiments include methods wherein said mesenchymal stem cell is activated with interleukin-23.

Preferred embodiments include methods wherein said mesenchymal stem cell is activated with interleukin-27.

Preferred embodiments include methods wherein said promoting agent is high density lipoprotein.

Preferred embodiments include methods wherein said promoting agent is BMP2.

Preferred embodiments include methods wherein said promoting agent is BMP4.

Preferred embodiments include methods wherein said promoting agent is BMP7.

Preferred embodiments include methods wherein said promoting agent is Klotho.

Preferred embodiments include methods wherein said promoting agent is GDF-11.

Preferred embodiments include methods wherein said promoting agent is GDF-15.

Preferred embodiments include methods wherein said promoting agent is amniotic fluid.

Preferred embodiments include methods wherein said promoting agent is umbilical cord blood plasma.

Preferred embodiments include methods wherein said promoting agent is BMP2.

Preferred embodiments include methods wherein said promoting agent suppresses NF-kappa B activation.

Preferred embodiments include methods wherein said promoting agent increases NRF2 activation.

Preferred embodiments include methods wherein said promoting agent increases heme-oxygenase-1 activation.

Preferred embodiments include methods wherein said promoting agent increases bcl-2 activation.

Preferred embodiments include methods wherein said promoting agent increases bcl-2XL activation.

Preferred embodiments include methods wherein said promoting agent increases survivin activation.

Preferred embodiments include methods wherein said promoting agent increases livin activation.

Preferred embodiments include methods wherein said promoting agent is an HDAC inhibitor.

Preferred embodiments include methods wherein said HDAC inhibitor is valproic acid.

Preferred embodiments include methods wherein said HDAC inhibitor is trichostatin A.

Preferred embodiments include methods wherein said HDAC inhibitor is sodium phenylbutyrate.

Preferred embodiments include methods wherein said HDAC inhibitor is butyrate.

Preferred embodiments include methods wherein said promoting agent is a GSK-3 inhibitor.

Preferred embodiments include methods wherein said GSK-3 inhibitor is lithium.

Preferred embodiments include methods wherein said promoting agent causes a change in the genotype of the thymic medullary epithelial cell population.

Preferred embodiments include methods wherein at least one promoting agent comprises or consists of an immortalizing oncogene.

Preferred embodiments include methods wherein said promoting agent is PIM1.

Preferred embodiments include methods wherein said promoting agent is SV40 large T antigen.

Preferred embodiments include methods wherein said promoting agent is abl1.

Preferred embodiments include methods wherein said promoting agent is AFF4.

Preferred embodiments include methods wherein said promoting agent is AKT2.

Preferred embodiments include methods wherein said promoting agent is AKL.

Preferred embodiments include methods wherein said promoting agent is AML1.

Preferred embodiments include methods wherein said promoting agent is MTG8.

Preferred embodiments include methods wherein said promoting agent is BCL6.

Preferred embodiments include methods wherein said promoting agent is MCF2.

Preferred embodiments include methods wherein said promoting agent is DCF3.

Preferred embodiments include methods wherein said promoting agent is EGFR.

Preferred embodiments include methods wherein said promoting agent is MLLT11.

Preferred embodiments include methods wherein said promoting agent is ERBB2.

Preferred embodiments include methods wherein said promoting agent is ETS1.

Preferred embodiments include methods wherein said promoting agent is CSF1R.

Preferred embodiments include methods wherein said promoting agent is FOS.

Preferred embodiments include methods wherein said promoting agent is FES.

Preferred embodiments include methods wherein said promoting agent is GNAS.

Preferred embodiments include methods wherein said promoting agent is HER2.

Preferred embodiments include methods wherein said promoting agent is FGF3.

Preferred embodiments include methods wherein said promoting agent is FGF4.

Preferred embodiments include methods wherein said promoting agent is JUN.

Preferred embodiments include methods wherein said promoting agent is c-kit.

Preferred embodiments include methods wherein said promoting agent is K-SAM.

Preferred embodiments include methods wherein said promoting agent is AKAP13.

Preferred embodiments include methods wherein said promoting agent is LCK.

Preferred embodiments include methods wherein said promoting agent is LM01.

Preferred embodiments include methods wherein said promoting agent is LYL1.

Preferred embodiments include methods wherein said promoting agent is MAS1.

Preferred embodiments include methods wherein said promoting agent is MDM2.

Preferred embodiments include methods wherein said promoting agent is MOS.

Preferred embodiments include methods wherein said promoting agent is MYH11.

Preferred embodiments include methods wherein said promoting agent is MYB.

Preferred embodiments include methods wherein said promoting agent is MYCN.

Preferred embodiments include methods wherein said promoting agent is PAX5.

Preferred embodiments include methods wherein said promoting agent is RAF.

Preferred embodiments include methods wherein said promoting agent is RAS.

Preferred embodiments include methods wherein said promoting agent is REL.

Preferred embodiments include methods wherein said promoting agent is ROS1.

Preferred embodiments include methods wherein said promoting agent is SKI (PDGF-BB).

Preferred embodiments include methods wherein said promoting agent is SET.

Preferred embodiments include methods wherein said promoting agent is SRC.

Preferred embodiments include methods wherein said promoting agent is TAL1.

Preferred embodiments include methods wherein said promoting agent is TAN1.

Preferred embodiments include methods wherein said promoting agent is TIAN.

Preferred embodiments include methods wherein said promoting agent is TSC2.

Preferred embodiments include methods wherein said promoting agent is TRK.

Preferred embodiments include methods wherein said promoting agent can be conditionally inactivated.

Preferred embodiments include methods wherein said promoting agent causes suppression of Foxn1 expression.

Preferred embodiments include methods wherein said promoting agent causes enhancement of AIRE expression.

Preferred embodiments include methods wherein thymic medullary epithelial cells are expanded in vitro by culture with one or more agents capable of suppressing expression of Foxn1.

Preferred embodiments include methods wherein said agent capable of suppressing expression of Foxn1 is an antisense oligonucleotide to Foxn1.

Preferred embodiments include methods wherein said antisense oligonucleotide to said Foxn1 is capable of suppressing expression of said Foxn1 through induction of RNase H activity.

Preferred embodiments include methods wherein said agent capable of suppressing expression of Foxn1 is a short interfering RNA molecule specific to Foxn1.

Preferred embodiments include methods wherein said agent capable of suppressing expression of Foxn1 is a short hairpin RNA molecule specific to Foxn1.

Preferred embodiments include methods wherein said agent capable of suppressing expression of Foxn1 is a hammerhead ribozyme specific to Foxn1.

Preferred embodiments include methods wherein said agent capable of suppressing expression of Foxn1 is n-acetylcysteine.

Preferred embodiments include methods wherein said agent capable of suppressing expression of Foxn1 is retinoic acid.

Preferred embodiments include methods wherein said agent capable of suppressing expression of Foxn1 is AAG-17.

Preferred embodiments include methods wherein said agent capable of suppressing expression of Foxn1 is CCL21.

Preferred embodiments include methods wherein said agent capable of suppressing expression of Foxn1 is CCL25.

Preferred embodiments include methods wherein said agent capable of suppressing expression of Foxn1 is NOTCH1.

Preferred embodiments include methods wherein said agent capable of suppressing expression of Foxn1 is BDNF.

Preferred embodiments include methods wherein said agent capable of suppressing expression of Foxn1 is NGF.

Preferred embodiments include methods wherein said agent capable of suppressing expression of Foxn1 is CNTF.

Preferred embodiments include methods wherein said agent capable of suppressing expression of Foxn1 is delta like 4.

Preferred embodiments include methods wherein said agent capable of suppressing expression of Foxn1 is EPCAM.

Preferred embodiments include methods wherein said agent capable of suppressing expression of Foxn1 is PLET1.

Preferred embodiments include methods wherein said agent capable of suppressing expression of Foxn1 is Tbx1.

Preferred embodiments include methods wherein said agent capable of suppressing expression of Foxn1 is Pax1.

Preferred embodiments include methods wherein said agent capable of suppressing expression of Foxn1 is Pax3.

Preferred embodiments include methods wherein said agent capable of suppressing expression of Foxn1 is Pax9.

Preferred embodiments include methods wherein said agent capable of suppressing expression of Foxn1 is Hoxa3.

Preferred embodiments include methods wherein said agent capable of suppressing expression of Foxn1 is Eya1.

Preferred embodiments include methods wherein said agent capable of suppressing expression of Foxn1 is Six1.

Preferred embodiments include methods wherein one or more antigens to which Treg cell generation is desired are placed in the combination of thymic medullary epithelial cells and pluripotent stem cells.

Preferred embodiments include methods wherein said antigen is administered in the form of antigenic peptides.

Preferred embodiments include methods wherein said antigenic peptides representing said antigen are determined by analysis of peptides capable of binding to HLA-1 alleles representing haplotype of the thymic donor.

Preferred embodiments include methods wherein said antigenic peptides representing said antigen are determined by analysis of peptides capable of binding to HLA-2 alleles representing haplotype of the thymic donor.

Preferred embodiments include methods wherein said antigenic peptides are designed based on transporter associated protein (TAP)-1 cleavage sites.

Preferred embodiments include methods wherein said antigens are autoantigens.

Preferred embodiments include methods wherein said antigens are alloantigens.

Preferred embodiments include methods wherein said antigens are xenoantigens.

Preferred embodiments include methods wherein said antigens are administered in the form of altered peptide ligands.

Preferred embodiments include methods wherein said antigens are administered together with tolerogenic dendritic cells.

Preferred embodiments include methods wherein tolerogenic dendritic cells are pulsed with said antigens and administered into a three-dimensional structure containing pluripotent stem cells and thymic medullary epithelial cells.

Preferred embodiments include methods wherein said tolerogenic dendritic cells are generated by culture of monocytes in GM-CSF.

Preferred embodiments include methods wherein said tolerogenic dendritic cells are generated by culture of monocytes in GM-CSF and low dose interleukin-4.

Preferred embodiments include methods wherein said tolerogenic dendritic cells are generated by culture of monocytes in GM-CSF and interleukin-10.

Preferred embodiments include methods wherein said tolerogenic dendritic cells are generated by culture of monocytes in GM-CSF and TGF-beta.

Preferred embodiments include methods wherein said tolerogenic dendritic cells are generated by culture of monocytes in GM-CSF and interleukin-13.

Preferred embodiments include methods wherein said tolerogenic dendritic cells are generated by culture of monocytes in GM-CSF and interleukin-20.

Preferred embodiments include methods wherein said tolerogenic dendritic cells are generated by culture of monocytes in GM-CSF and interleukin-22.

Preferred embodiments include methods wherein said tolerogenic dendritic cells are generated by culture of monocytes in GM-CSF and interleukin-35.

Preferred embodiments include methods wherein said tolerogenic dendritic cells are generated by culture of monocytes in GM-CSF and interleukin-38.

Preferred embodiments include methods wherein said tolerogenic dendritic cells are generated by culture of monocytes in GM-CSF and soluble HLA-G.

Preferred embodiments include methods wherein said tolerogenic dendritic cells are generated by culture of monocytes in GM-CSF and siRNA to interleukin-12 p35.

Preferred embodiments include methods wherein said tolerogenic dendritic cells are generated by culture of monocytes in GM-CSF and one or more inhibitors of NF-kappa B.

Preferred embodiments include methods wherein said inhibitor of NF-kappa B is doxycycline.

Preferred embodiments include methods wherein said inhibitor of NF-kappa B is minocycline.

Preferred embodiments include methods wherein said inhibitor of NF-kappa B is MG-132.

Preferred embodiments include methods wherein said inhibitor of NF-kappa B is caffeic acid.

Preferred embodiments include methods wherein said inhibitor of NF-kappa B is triptolide.

Preferred embodiments include methods wherein said inhibitor of NF-kappa B is arctigenin.

Preferred embodiments include methods wherein said inhibitor of NF-kappa B is vascular endothelial growth factor.

Preferred embodiments include methods wherein said inhibitor of NF-kappa B is thymoquinone.

Preferred embodiments include methods wherein said inhibitor of NF-kappa B is sulforaphane.

Preferred embodiments include methods wherein said inhibitor of NF-kappa B is hydrogen sulfide.

Preferred embodiments include methods wherein said inhibitor of NF-kappa B is IKK16.

Preferred embodiments include methods wherein said inhibitor of NF-kappa B is TPCA1.

Preferred embodiments include methods wherein said inhibitor of NF-kappa B is withaferin A.

Preferred embodiments include methods wherein said inhibitor of NF-kappa B is celastrol.

Preferred embodiments include methods wherein said inhibitor of NF-kappa B is sulfasalazine.

Preferred embodiments include methods wherein said inhibitor of NF-kappa B is zoledronic acid.

Preferred embodiments include methods wherein said inhibitor of NF-kappa B is FPS ZM!.

Preferred embodiments include methods wherein said inhibitor of NF-kappa B is piceatannol.

Preferred embodiments include methods wherein said inhibitor of NF-kappa B is cardamonin.

Preferred embodiments include methods wherein said inhibitor of NF-kappa B is luteolin.

Preferred embodiments include methods wherein said inhibitor of NF-kappa B is honokiol.

Preferred embodiments include methods wherein said inhibitor of NF-kappa B is amlexanox.

Preferred embodiments include methods wherein said pluripotent stem cells are combined with said thymic medullary epithelial cells in the presence of a scaffold to facilitate such interaction.

Preferred embodiments include methods wherein said scaffold is comprised of decellularized thymic tissue.

Preferred embodiments include methods wherein said decellularized thymic tissue is produced by treatment of thymic tissue with one or more acidification agents.

Preferred embodiments include methods wherein said decellularized thymic tissue is produced by treatment of thymic tissue with polyethylene glycol.

Preferred embodiments include methods wherein said decellularized thymic tissue is produced by treatment of thymic tissue with a surfactant.

Preferred embodiments include methods wherein said decellularized thymic tissue is produced by treatment of thymic tissue with sodium dodecyl sulfate.

Preferred embodiments include methods wherein said decellularized thymic tissue is produced by treatment of thymic tissue with Triton X-100.

Preferred embodiments include methods wherein said decellularized thymic tissue is produced by treatment of thymic tissue with Sodium deoxycholate.

Preferred embodiments include methods wherein said decellularized thymic tissue is produced by treatment of thymic tissue with Zwitterionic CHAPS.

Preferred embodiments include methods wherein said decellularized thymic tissue is produced by treatment of thymic tissue with Peracetic acid.

Preferred embodiments include methods wherein said decellularized thymic tissue is produced by treatment of thymic tissue with Ethylenediaminetetraacetic acid.

Preferred embodiments include methods wherein said decellularized thymic tissue is produced by treatment of thymic tissue with trypsin.

Preferred embodiments include methods wherein said decellularized thymic tissue is produced by treatment of thymic tissue with trypsin and DNAse.

Preferred embodiments include methods wherein said decellularized thymic tissue is produced by treatment of thymic tissue with trypsin and RNAse.

Preferred embodiments include methods wherein said decellularized thymic tissue is produced by treatment of thymic tissue with trypsin, RNAse and DNAse.

Preferred embodiments include methods wherein said decellularized thymic tissue is produced by treatment of thymic tissue with supercritical carbon dioxide

Preferred embodiments include methods wherein said scaffold possesses a pharmaceutically acceptable carrier with the ability of serving a scaffolding function, and wherein said pharmacological carrier is collagen II.

Preferred embodiments include methods wherein said pharmaceutically acceptable carrier serving a scaffolding function is decellularized placental tissue.

Preferred embodiments include methods wherein said pharmaceutically acceptable carrier serving a scaffolding function is Matrigel.

Preferred embodiments include methods wherein said pharmaceutically acceptable carrier serving a scaffolding function is collagen II.

Preferred embodiments include methods wherein said pharmaceutically acceptable carrier serving a scaffolding function is small intestinal submucosa.

Preferred embodiments include methods wherein said pharmaceutically acceptable carrier serving a scaffolding function is fibronectin.

Preferred embodiments include methods wherein said pharmaceutically acceptable carrier serving a scaffolding function is thrombospondin.

Preferred embodiments include methods wherein said pharmaceutically acceptable carrier serving a scaffolding function is parlecan.

Preferred embodiments include methods wherein said pharmaceutically acceptable carrier serving a scaffolding function is brevican.

Preferred embodiments include methods wherein said pharmaceutically acceptable carrier serving a scaffolding function is type IV collagen.

Preferred embodiments include methods wherein said pharmaceutically acceptable carrier serving a scaffolding function is tenascin.

Preferred embodiments include methods wherein said pharmaceutically acceptable carrier serving a scaffolding function is elastin.

Preferred embodiments include methods wherein said pharmaceutically acceptable carrier serving a scaffolding function is fibronectin.

Preferred embodiments include methods wherein amniotic fluid and pharmaceutically acceptable carrier have a weight ratio in the range between 1:10 and 10:1 are utilized as the basis of the scaffold.

Preferred embodiments include methods wherein the pharmaceutically acceptable carrier is Wharton's jelly, with optionally; a) a biocompatible gelation agent; b) a polysaccharide; c) collagen IV; d) high molecular weight hyaluronic acid; e) poly-1-lysine; f) or platelet rich plasma.

Preferred embodiments include methods wherein an inhibitor of regenerative cell death is admixed.

Preferred embodiments include methods wherein said inhibitor of regenerative cell death is one or more agents that induce activation of NF-kappa B.

Preferred embodiments include methods wherein said activator of said NF-kappa B is oxytocin.

Preferred embodiments include methods wherein said activator of said NF-kappa B is beta glucan.

Preferred embodiments include methods wherein said activator of said NF-kappa B is poly IC.

Preferred embodiments include methods wherein said activator of said NF-kappa B is poly IC:LC.

Preferred embodiments include methods wherein said activator of said NF-kappa B is CpG DNA.

Preferred embodiments include methods wherein said activator of said NF-kappa B is beta glucan.

Preferred embodiments include methods wherein said agent that induces activation of NF-kappa B cells is apoptotic.

Preferred embodiments include methods wherein said apoptotic bodies are derived from monocytes.

Preferred embodiments include methods wherein said monocytes are adherent to fibronectin.

Preferred embodiments include methods wherein said monocytes express CD14.

Preferred embodiments include methods wherein said monocytes express CD69.

Preferred embodiments include methods wherein said monocytes express CD16.

Preferred embodiments include methods wherein said monocytes express CD73.

Preferred embodiments include methods wherein said monocytes express CD105.

Preferred embodiments include methods wherein said monocytes are activated prior to induction of apoptosis

Preferred embodiments include methods wherein said activation of monocytes is performed in the presence of interleukin-4.

Preferred embodiments include methods wherein said activation of monocytes is performed in the presence of interleukin-13.

Preferred embodiments include methods wherein said activation of monocytes is performed in the presence of interleukin-10.

Preferred embodiments include methods wherein said activation of monocytes is performed in the presence of TNF-alpha.

Preferred embodiments include methods wherein said activation of monocytes is performed in the presence of lymphotoxin.

Preferred embodiments include methods wherein said activation of monocytes is performed in the presence of LIGHT.

### DETAILED DESCRIPTION OF THE INVENTION

The invention discloses processes, means, and compositions of matter of generation of Tregs through the recreation or recapitulation of the natural process of Treg development. In one embodiment the invention teaches the placement of a pluripotent stem cell population into contact with a thymic derived medullary epithelial cell population in a manner to facilitate generation of T regulatory cells. The cell to cell contact is ideally created in a three dimensional context such as cells being grown in a spheroid or suspension culture. Optionally desired antigens are placed into the growing pluripotent stem cell-thymic medullary epithelial cell mixture if antigen-specific T cells are to be generated. Furthermore, in some embodiments the invention provides addition of various cytokines and/or growth factors and/or morphogens in order to accelerate Treg generation.

“Adaptive immunity” is described as T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

“T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

“Autoimmune disease” or “autoimmune response” is a response in which the immune system of an individual initiates and may propagate a primary and/or secondary response against its own tissues or cells. An “alloimmune response” is one in which the immune system of an individual initiates and may propagate a primary and/or secondary response against the tissues, cells, or molecules of another, as, for example, in a transplant or transfusion.

The term “cell-mediated immunity” refers to (1) the recognition and/or killing of virus and virus-infected cells by leukocytes and (2) the production of different soluble factors (cytokines) by these cells when stimulated by virus or virus-infected cells. Cytotoxic T lymphocytes (CTLs), natural killer (NK) cells and antiviral macrophages are leukocytes that can recognize and kill virus-infected cells. Helper T cells can recognize virus-infected cells and produce a number of important cytokines. Cytokines produced by monocytes (monokines), T cells, and NK cells (lymphokines) play important roles in regulating immune functions and developing antiviral immune functions. A host T cell response can be directed against cells of the host, as in autoimmune disease. For example, the T cells in type I diabetes (T1D) recognize an “antigen” that is expressed by the host, which causes the destruction of normal host cells—for T1D, the endocrine cells of the islets of Langerhans of the pancreas. A T cell response may also occur within a host that has received a graft of foreign cells, as is the case in graft-versus-host disease (GVHD) in which T cells from the graft attack the cells of the host, or in the case of graft rejection in which T cells of the host attack the graft.

“T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by myeloid-derived suppressor cells (MDSC) activity. Several Treg subsets have been identified that can inhibit autoimmune and chronic inflammatory responses and to maintain immune tolerance in tumor-bearing hosts. These subsets include interleukin 10-(IL-10-) secreting T regulatory type 1 (Tr1) cells, transforming growth factor-.beta.-(TGF-.beta.-), secreting T helper type 3 (Th3) cells, and “natural” CD4.sup.+/CD25.sup.+ Tregs (Trn).

The phrase “inducing T regulatory cells” means activation, amplification, and generation of Tregs to inhibit or reduce the T cell response. One method of induction is through the use of the MDSCs.

The phrase “T cell tolerance” refers to the anergy (non-responsiveness) of T cells when presented with an antigen. T cell tolerance prevents a T cell response even in the presence of an antigen that existing memory T cells recognize.

“Differentiate” refers to the genetic process by which cells are produced with a specialized phenotype. A differentiated cell of any type has attained all of the characteristics that define that cell type. This is true even in the progression of cell types. For example, if cell type X matures to cell type Y which then overall matures to cell type Z, an X cell differentiates to a Y cell when it has attained all the characteristics that define a type Y cell, even though the cell has not completely differentiated into a type Z cell.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V.sub.H) and a heavy chain constant region. The heavy chain constant region is comprised of three domains, C.sub.H1, C.sub.H2 and C.sub.H3. Each light chain is comprised of a light chain variable region (abbreviated herein as V.sub.L) and a light chain constant region. The light chain constant region is comprised of one domain, C.sub.L. The V.sub.H and V.sub.L regions can be further subdivided into regions of hypervariability, termed complementarity determining regions (CDR), interspersed with regions that are more conserved, termed framework regions (FR). Each V.sub.H and V.sub.L is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

“Cytokine” is a generic term for a group of proteins released by one cell population which act on another cell population as intercellular mediators. Examples of such cytokines are lymphokines, monokines, and traditional polypeptide hormones. Included among the cytokines are interferons (IFN, notably IFN-.gamma.), interleukins (IL, notably IL-1, IL-2, IL-4, IL-10, IL-12), colony stimulating factors (CSF), macrophage colony stimulating factor (M-CSF), granulocyte macrophage colony stimulating factor (GM-CSF), thrombopoietin (TPO), erythropoietin (EPO), leukemia inhibitory factor (LIF), kit-ligand, growth hormones (GH), insulin-like growth factors (IGF), parathyroid hormone, thyroxine, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), mullerian-inhibiting substance, mouse gonadotropin-associated peptide, inhibin, activin, vascular endothelial growth factor (VEGF), integrin, nerve growth factors (NGF), platelet growth factor, transforming growth factors (TGF), osteoinductive factors, etc. Those of particular interest for the present invention include IFN-.gamma., IL-10, and TGF-.beta.

“Autoantigen” refers to a molecule that is endogenous to a cell or organism that induces an autoimmune response.

“Transplant rejection” means that a transplant of tissue or cells is not tolerated by a host individual. The transplant is not tolerated in that it is attacked by the host's own immune system or is otherwise not supported by the host. The transplant may be an allotransplant, a transplant of tissue or cells from another individual of the same species, or an autotransplant, a transplant of the host's own tissue or cells. Transplant rejection encompasses the rejection of fluids through transfusion.

“Induced pluripotent stem cells” commonly abbreviated as iPS cells or iPSCs, refers to a type of pluripotent stem cell artificially generated from a non-pluripotent cell, typically an adult somatic cell, or terminally differentiated cell, such as fibroblast, a hematopoietic cell, a myocyte, a neuron, an epidermal cell, or the like.

“Differentiation” and “cell differentiation” refer to a process by which a less specialized cell (i.e., stem cell) develops or matures or differentiates to possess a more distinct form and/or function into a more specialized cell or differentiated cell, (i.e., thymic epithelial cell).

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

In one embodiment of the invention, Treg cells are generated by coculture of pluripotent stem cells with thymic medullary epithelial cells in spheroid culture conditions. This process, alone or together with various Treg adjuvants generated populations of Treg cells which may be enriched and expanded. For the practice of the invention, in one embodiment, an “enriched Foxp3′ sample” or “lymphoblast population” refers to those samples that have been enriched for Foxp3′ cells by selection of cells based various flow cytometric identified markers and/or size/density markers. For example, isolation of enriched FoxP3 expressing cells may be performed using forward scatter (FSC) and/or side scatter (SSC), or any combination of these properties with cell surface markers, for example CD4 and/or CD25/and/or CTLA4. An enriched sample is one in which more than 75%, 80%, 85%, 90%, 95%, 99% or more cells express Foxp3. A “non-lymphoblast population”, which is deemed to be contamination and not useful for the purpose of the invention refers to those samples based on FSC and/or SSC, or any combination of these properties with cell surface markers, for example CD4 and/or CD25 wherein less than 80% of the cells express Foxp3. In the practice of the invention, the aggregate of pluripotent stem cells and thymic medullary epithelial cells is cultured in vitro. There are numerous culture techniques that are applicable including “stirrer” cultures, hollow-fiber cultures, and bioreactors. In some embodiments the cultures of pluripotent stem cells and thymic medullary epithelial cells are performed in the presence of stromal cells. Various types of stromal cells may be utilized including fibroblastic-like cells, lymph node associated stromal cells, endothelial cells, and mesenchymal stem cells. In some situations, extracellular vimentin expressing mesenchymal stem cells are utilized for culture together with thymic medullary epithelial cells. Once sufficient cells are grown, the isolation of Treg cells is performed using various means known in the art. Methods for detecting Foxp3 expression in isolated cell populations are known to those of skill in the art, and include, without limitation, intracellular staining with anti-Foxp3 reagents, for example, antibodies and nucleic acid probes. In one embodiment “cell sorters”, are utilized. These are a type of flow cytometers which have the ability to selectively deposit cells from particular populations into tubes, or other collection vessels. It is known that for the sorting of cells, the instruments electronics interprets the signals collected for each cell as it is interrogated by the laser beam and compares the signal with sorting criteria set on the computer, the gate. If the cell meets the required criteria, an electrical charge is applied to the liquid stream which is being accurately broken into droplets containing the cells. This charge is applied to the stream at the precise moment the cell of interest is about to break off from the stream, then removed when the charged droplet has broken from the stream. As the droplets fall, they pass between two metal plates, which are strongly positively or negatively charged. Charged droplets get drawn towards the metal plate of the opposite polarity, and deposited in the collection vessel, or onto a microscope slide, for further examination. The cells can automatically be deposited in collection vessels as single cells or as a plurality of cells, e.g. using a laser, e.g. an argon laser (488 nm) and for example with a flow cytometer fitted with an Autoclone unit (Coulter EPICS Altra, Beckman-Coulter, Miami, Fla., USA). Other examples of suitable FACS machines useful for the methods of the invention include, but are not limited to, MoFlo™. High-speed cell sorter (Dako-Cytomation Ltd), FACS Aria™ (Becton Dickinson), ALTRA™. Hyper sort (Beckman Coulter) and CyFlow™ sorting system (Partec GmbH). Other methods and means of isolating Treg cells are with nucleic acid encoding green fluorescent protein (GFP) being inserted into the Foxp3 gene through a knock-in procedure. The population of cells with 80% or more of the cells expressing GFP with the relatively highest FSC and relatively lowest SSC identify the Foxp3+ lymphoblast population.

In some embodiments, the pluripotent stem cell derived lymphoblast population is isolated from Tregs expanded in vitro. Tregs are thymus derived regulatory T cells and can be isolated from lymphoid tissues, for example, the thymus, spleen, lymph nodes or from bodily fluids, for example, blood. Tregs can be obtained from any mammalian subject, including mice and humans. Methods for expanding Tregs are known in the art. Such methods often entail, without limitation, the culturing of a starting population of isolated cells under appropriate culture conditions. Often the isolated cells are cells that are CD4+ and/or CD25+.

In some embodiments, the lymphoblast population and/or the non-lymphoblast populations develop and can be isolated by expanding isolated CD4+CD25+ in the presence of anti-CD3, anti-CD28, and/or IL-2. These cells can be maintained in vitro over the course of 1, 2, 3, 4, 5, 6, 7, or more days. Cells resulting from this process are referred to as “expanded Tregs.” In some embodiments, the lymphoblast population is isolated from Treg. In some embodiments, the lymphoblast population and/or the non-lymphoblast populations develop and can be isolated by culturing isolated CD4+ cells in the presence of anti-CD3, anti-CD28, IL-2, and/or TGF-β. Cells resulting from this process are referred to as “Tregs.” Populations of enriched Foxp3+ cells, by methods of such as FACS, can be used in the treatment of diseases and disorders. In certain aspects, the present invention provides methods and compositions for the prevention and treatment of immune conditions; that is, those diseases, disorders and reactions or responses wherein the immune system contributes to pathogenesis. As used herein, the term “sample” or “biological sample” refers to tissues or bodily fluids removed from a mammal, preferably human, and which contain regulatory T cells. In some embodiments, the samples are taken from individuals with an immune response which needs to be suppressed. In some embodiments, the individual has an allergy, Graft vs. Host Disease, an organ transplant, or autoimmune disorder. Samples preferably are blood and blood fractions, including peripheral blood. The biological sample is drawn from the body of a mammal, such as a human, and may be blood, bone marrow cells, or similar tissues or cells from an organ afflicted with the unwanted immune response. Methods for obtaining such samples are well known to workers in the fields of cellular immunology and surgery. They include sampling blood in well-known ways or obtaining biopsies from the bone marrow or other tissue or organ. In preferred embodiments, the sample is a T-cell enriched sample in which the sample cells are substantially T-cells. Immune conditions also include autoimmunity. Autoimmunity is the persistent and progressive immune reactions to noninfectious self-antigens, as distinct from infectious non self-antigens from bacterial, viral, fungal, or parasitic organisms which invade and persist within mammals and humans. The treatment modalities disclosed herein include diseases associated with the inability of the immune system to discriminate between self and non-self. Examples of autoimmune diseases include, without limitation, immune dysregulation, polyendocrinopathy, enteropathy, X-linked (IPEX), type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

The therapies disclosed in the current invention revolve around the fact that the thymus is the principal site of Treg development, providing the microenvironments required to support T-cell differentiation and repertoire selection. These unique processes are dependent on the thymic stroma. This comprises a highly ordered, three-dimensional network of thymic epithelial cells interspersed with non-epithelial stromal cell-types and is organized into two main compartments, the cortex and the medulla, which each contain several distinct epithelial subpopulations. The different thymic epithelial cell-types are thought to provide specific molecular niches required for different stages of thymocyte differentiation and maturation, as T-cell development requires interactions with multiple epithelial subpopulations. Accordingly, in some embodiments of the invention thymi extracted from cadaveric donors are used decellularized and seeded with pluripotent stem cells and/or mesenchymal stem cells. The invention aims to recapitulate the embryonic process of Treg formation. During embryogenesis, contributions from the 3rd pharyngeal pouch endoderm, neural crest-derived mesenchyme and possibly the 3rd pharyngeal cleft ectoderm are required for formation of the thymic primordium, which is first present as a discrete organ at day 12.5 of murine embryonic development (E12.5). In the present application, the inventors address the need for a method to allow the generation of Treg cells and disclose uses of TEPCs and compositions containing them. Based on their in vivo properties, TEPCs are an ideal material for use in transplantation therapy or for in vitro thymi generation. They are an expandable cell-type, capable of producing all major mature thymic epithelial sub-populations. In vitro however they have proven difficult to maintain in culture. The present disclosure therefore provides materials and methods for enriching TEPC populations, for improving the viability of an isolated TEPC, for expanding a population of TEPCs in vitro, and for causing or allowing TEPCs to differentiate into cortical and medullary thymic epithelial cell-types to generate a functional thymus in vitro or in vivo. This strategy circumvents both ethical and practical issues surrounding the use in culture or for transplantation of cells obtained directly from human fetal tissue. In particular, each fetus provides only a small number of cells, insufficient for clinical purposes. In a first aspect, the invention provides a method for improving the viability of a population of isolated thymic epithelial progenitor cells (TEPCs), which method comprises contacting the cells, or one or more ancestors thereof, with at least one viability promoting agent. By “improving the viability of a population of isolated thymic epithelial progenitor cells (TEPCs)” is meant that the rate of decline of the number of viable TEPCs in the population is reduced. This may include the number of viable TEPCs in the population being maintained at a substantially constant level, or the number of viable TEPCs in the population being increased over time.

In preferred embodiments, Treg cells are generated from culture of pluripotent stem cells with one or more viability promoting agents induces or enhances TEPC replication and enhance Treg formation. In this way, the decline in the viability of the TEPC population as a whole may, at least in part, be slowed, arrested or reversed, by the production of daughter cells from an original group of TEPCs. If a replicating population of cells approaches confluence, then the population may be sub-divided into two or more daughter populations. Each population may be diluted in a suitable medium, as discussed elsewhere herein. The one or more viability promoting agents may be protein, polypeptide, glycoprotein, proteoglycan, carbohydrate, oligosaccharide, polysaccharide, nucleotide, oligonucleotide or nucleic acid in nature. The agent may be selected from the group consisting of a hormone, growth factor, cytokine, steroid, interferon, colony stimulating factor, extracellular matrix material. It may be produced by a specific cell-type or cell-types, and may be a cell surface agent and/or an agent secreted into the culture supernatant of those cells. Specific examples of suitable agents include insulin-like growth factor 1 IGF-1, epidermal growth factor EGF, insulin, hydrocortisone, transferrin, high density lipoprotein (HDL), bone morphogenetic protein (BMP2)2, (BMP)4 and (BMP)7 noggin, fibroblast growth factor 1 (Fgf1), Fgf2, Fgf3, Fgf8, and sonic hedgehog (shh). The one or more viability promoting agents may be added continually or periodically to the TEPC population. Alternatively, there may be a single, initial period of exposure to the one or more agents, which period can involve a single addition of the one or more agents or a plurality of successive additions. Where a TEPC population is contacted with one or more viability promoting agents on a number of consecutive occasions, the agent or agents added on each occasion may be different from those added on a previous occasion. Where the TEPC population is subjected to a single, initial period of exposure, the one or more agents may cause the cells to undergo a long-term physiological change. That change may enable the viability of the population of TEPCs to be substantially improved without the need for a subsequent addition of any further viability promoting agents. In certain embodiments of the invention, the one or more viability promoting agents may cause a change in the genotype of at least one TEPC in the population. This change in genotype may improve the viability of the TEPC, e.g. by transforming the TEPC into an immortalized or reversibly immortalized state. In this connection, the one or more viability promoting agents may include at least one polynucleotide. The polynucleotide may be part of a vector which may be plasmid or viral or artificial chromosome. Suitable vectors can be chosen or constructed, containing appropriate regulatory sequences, e.g. promoter sequences, terminator fragments, polyadenylation sequences, enhancer sequences. Vectors may contain selectable marker genes and other sequences as appropriate. Marker genes such as antibiotic resistance or sensitivity genes, or fluorescent- or epitope-tagged proteins may be used in identifying clones containing nucleic acid of interest, as is well known in the art. Clones may also be identified or further investigated by binding studies, e.g. by Southern blot hybridisation. Inside the TEPC, the nucleic acid comprising the polynucleotide may exist as an isolated extra-genomic sequence, or it may integrate, preferably stably, into the host cell genome. As an isolated sequence, it may be capable of replication, e.g. as an episome or artificial chromosome. Integration may be promoted by including in the nucleic acid sequences which promote recombination with the genome, in accordance with standard techniques. The nucleic acid may include sequences which direct its integration to a particular site in the genome where a coding sequence contained within it falls under the control of regulatory elements able to drive and/or control expression of that sequence in the TEPC.

In one embodiment Treg are generated to treat type 1 diabetes. This may be performed by creating said Treg using an artificial thymus in vitro, the cells are cultured in a nutritive medium which may additionally comprise one or more other cell-types, e.g. non-epithelial cells of the thymic stroma, mesenchymal cells, cells of the vascular endothelium, haematopoietic stem cells/lymphoid progenitor cells. The cells may be grown on a solid support matrix. Production of a functional artificial thymus may be detected by the ability of the thymus to cause differentiation of haematopoietic stem cells (HSCs) and/or lymphoid progenitor cells to mature CD4+ or CD8+ T cells. Mature T cells may be detected using labeled antibodies against CD4 or CD8, e.g. by using microscopy or flow cytometry, as described elsewhere herein. The invention therefore provides a method of generating an artificial thymus in vitro. The method comprises providing a population of cortical and medullary thymic epithelial cells, which population has been obtained by causing or allowing differentiation of a population of isolated TEPCs. The method may comprise inducing said differentiation by contacting the TEPCs with one or more factors supplied by or derived from mesenchymal cells, HSCs, lymphoid progenitors, thymocytes, vascular endothelial cells, or mixtures of such cells. The method may comprise co-culturing TEPCs with one or more of said cells. The invention also extends to a method of producing Treg that possess features of inducible Treg, which method comprises contacting HSCs and/or lymphoid progenitors/thymocytes, with an artificial thymus of the invention. HSCs and lymphoid progenitors may be obtained from blood or bone marrow using standard techniques well known to those skilled in the art, e.g. by biopsy followed by e.g. FACS, affinity purification, using antibodies directed to appropriate cell markers. Such techniques may also be used to obtain the mature T-cells from the artificial thymus. In certain embodiments of the invention, the TEPCs of the artificial thymus may be derived from two or more different individuals or two or more species. In this way, the mature T-cells produced by the thymus may be tolerant to the tissues of two or more individuals or species. This may have beneficial consequences if the T-cells are for use in allo or xeno-graft patients: the T-cells may be tolerant to both graft and host. A further option is to establish a bank of cells covering a range of immunological compatibilities from which an appropriate choice can be made for an individual patient. TEPCs cells derived from one individual may also be altered to ameliorate rejection when they or their progeny are introduced into a second individual. By way of example, one or more MHC alleles in a donor cell may be replaced with those of a recipient, e.g. by homologous recombination, or augmented with those of a recipient, or donor e.g. by additive transgenesis. Further aspects of the present invention include a Treg cells produced by the TEPC-derived artificial thymus, and a composition, medicament or drug containing such a T-cell. The invention also provides the use of such a T-cell or composition in a method of medical treatment, e.g. to restore cellular immunity, and the use of such a T-cell for the manufacture of a medicament. Formulation and administration of pharmaceutical compositions is described elsewhere herein.

For tolerogenesis in type 1 diabetes, in certain embodiments of this invention, two, three, or a higher plurality of tolerogens are used in the reaction between pluripotent stem cells and thymic medullary epithelial cells in order to generate Tregs. It may be desirable to implement these embodiments when there is a plurality of target antigens. It may also be desirable to provide a cocktail of antigens to cover several possible alternative targets. For example, a cocktail of histocompatibility antigen fragments could be used to tolerize a subject in anticipation of future transplantation with an allograft of unknown phenotype. In another example, a mixture of allergens may serve as inducing antigen for the treatment of atopy. Tolerogens can be prepared by a number of techniques known in the art, depending on the nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

In certain embodiments of this invention, the tolerogen is administered together with the Treg. Said tolerogen comprises a complex mixture of antigens obtained from a cell or tissue, one or more of which plays the role of tolerogen. The tolerogens may be in the form of whole cells, either intact or treated with a fixative such as formaldehyde, glutaraldehyde, or alcohol; in the form of a cell lysate, created by detergent solubilization or mechanical rupture of cells or tissue, followed by clarification. The tolerogens may also be obtained by subcellular fractionation, particularly an enrichment of plasma membrane by techniques such as differential centrifugation, optionally followed by detergent solubilization and dialysis. Other separation techniques are also suitable, such as affinity or ion exchange chromatography of solubilized membrane proteins. Mixtures of antigens from cells or tissues are of particular interest in a number of applications of this invention. For example, for the treatment of organ-specific autoimmune disease, where the identity of the target antigen is unknown, or to provide a plurality of antigens to heighten the tolerogenic response. Suitable sources of cells for this purpose would be a biopsy sample of the same tissue from the subject to be treated, or a cultured cell line of the same tissue type. To tolerize a recipient to a planned tissue graft, the cell source is preferably obtained from either the donor or an individual sharing at least one major histocompatibility complex allotype with the donor. In humans, preferably two or more allotypes are shared at the HLA-A/B and HLA-DR locus (in order of increasing preference in the treatment of graft rejection; in the order of decreasing preference in the treatment of graft-versus-host disease). For tolerization against histocompatibility class II antigens (the usual target of an acute allograft rejection), peripheral blood mononuclear cells, spleen cells or lymph node cells are particularly appropriate. For tolerization against carbohydrate antigens (the usual target of hyperacute xenograft rejection), it is appropriate to use any cell type that is enriched at the target, such as endothelial cells or leukocytes.

For use in the methods of the invention, it is desirable to isolate populations of tolerogenic dendritic cells and utilize to stimulate Tregs in vivo or ex vivo. Furthermore, the invention disclosed combinations of tolerogenic dendritic cell administration together with Treg administration. Separation by cell staining may use conventional methods, as known in the art, including magnetic bead separation, affinity selection, fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). The affinity reagents may be antibodies, or other specific receptors or ligands for the cell surface molecule CCR9, CMKLR1 or CD103, which are optionally used in combination with reagents specific for one or both of CD11c and B220 in mice (gated on CD3/CD19 negative cells); and Lin-1 (i.e. non-DC lineage markers such as one or more of CD3, CD14, CD16, CD19, CD20, CD56), CD11c and CD123 in humans. The cells may be isolated from lymphoid tissue, from blood, or from in vitro culture, e.g. bone marrow culture, etc. In addition to antibody reagents, polynucleotide probes specific for an mRNA of interest, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker. The antibodies are added to cells, and incubated for a period of time sufficient to bind the available antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc. The cells of interest may be separated from a complex mixture of cells by techniques that enrich for cells having the above described characteristics. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum. Compositions highly enriched for tolerogenic dendritic cells are achieved in this manner. The subject population may be at or about 50% or more of the cell composition, and preferably be at or about 75% or more of the cell composition, and may be 90% or more. The desired cells are identified by their surface phenotype, by the ability to induce tolerance, etc. The enriched cell population may be used immediately, or may be frozen at liquid nitrogen temperatures and stored for long periods of time, being thawed and capable of being reused. The cells will usually be stored in 10% DMSO, 50% FCS, 40% RPMI 1640 medium. The population of cells enriched for tolerogenic dendritic cells may be used in a variety of screening assays and cultures, as described below.

The enriched tolerogenic dendritic cells population may be grown in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive such as Flt3L and thrombopoietin. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. A wide variety of growth factors may be used in culturing the cells. Optionally, cofactors such as all-trans retinoic acid to induce CCR9 expression or 1a, 25 dihydroxyvitamin D3 and/or dexamethasone or other steroid-based agents known to be immunosuppressive, are included in expansion culture medium at a concentration sufficient to increase the number of tolerogenic DC populations in in vitro cultures. In addition to, or instead of growth factors, the subject cells may also be grown in a co-culture with fibroblasts, stromal or other feeder layer cells. The tolerogenic dendritic cells may find use in methods of inducing tolerance. Various routes and regimens for delivery may be used, as known and practiced in the art. The dose of cells may be from about 104-109 per dose, depending on the size of the animal and the tolerogen. Administration may be at a localized site, e.g. sub-cutaneous, or systemic, e.g. intraperitoneal, intravenous, etc. Tolerogenic formulations will typically contain from about 0.1 μg to 1000 μg, more preferably 1 μg to 100 μg, of the selected tolerogen, while in embodiments where the dendritic cells are derived from a graft donor, no exogenous tolerogen is required. The tolerogen composition may additionally contain biological buffers, excipients, preservatives, and the like. The dendritic cells may be pulsed with tolerogen prior to administration, e.g. by suspending the dendritic cells in a solution of the tolerogen, followed by washing the cells, prior to administration. If desired, the cells may be administered in several doses, e.g. twice weekly, weekly, monthly, etc., for a period of time sufficient to induce long-term tolerance. The cells may be administered in any physiologically acceptable medium, normally intravascularly, although they may also be introduced into any convenient site, where the cells may find an appropriate site for tolerization. Usually, at least 1×105 cells will be administered, preferably 1×106; 107, 108 or more. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with dendritic cell proliferation and differentiation. Conditions of interest for treatment include preparation for allogeneic transplantation, where the dendritic cells have at least one MHC allele in common with the cells to be transplanted, which MHC allele is normally present in the recipient. For example, a human recipient that is matched with a tissue, organ or cell at 4 out of 5 HLA A, B and C alleles may be tolerized with dendritic cells that bear the two unmatched alleles. In this way the recipient is made tolerant of all HLA alleles present in the engrafted cells.

Genes may be introduced into the Treg cells for a variety of purposes, e.g. replace genes having a loss of function mutation, provide recognition of a particular antigen, suppress activation of a particular antigen receptor, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to transfect the target cells, e.g. electroporation, calcium precipitated DNA, fusion, transfection, lipofection and the like. The particular manner in which the DNA is introduced is not critical to the practice of the invention. In some embodiments Tregs and/or tolerogenic DC are generated from pluripotent stem cells. Pluripotent stem cells including inducible pluripotent stem cells, embryonic stem cells, and somatic cell nuclear transfer derived stem cells. Pluripotent stem cells are characterized by marker expression. One marker useful for identification and characterization of pluripotent stem cells is the SSEA family of markers. SSEAs were originally identified by three monoclonal antibodies recognizing defined carbohydrate epitopes associated with the lacto- and globo-series glycolipids SSEA-1, SSEA-3, and SSEA-4. These carbohydrate-associated molecules are involved in controlling cell surface interactions during development. SSEA-1 (CD15/Lewis x) is expressed on the surface of murine embryos at the pre-implantation stage, as well as in mouse and human germ cells, and on the surface of teratocarcinoma stem cells, but it is absent in human ESC and human embryonic carcinoma cells. SSEA-1 expression was also found in the oviduct epithelium, endometrium, and epididymis, as well as some areas of the brain and kidney tubules in adults. SSEA-1 expression increases upon differentiation in human cells, decreases in differentiation in mouse. SSEA-3 and SSEA-4 are synthesized during oogenesis and are present in the membranes of oocytes, zygotes, and early cleavage-stage embryos. They are expressed in undifferentiated primate ESC, human embryonic germ (EG) cells, human teratocarcinoma stem cells, and ESC. Other markers useful for characterization of pluripotent stem cells are the cluster of differentiation (CD) antigens which are surface proteins that belong to several different classes, such as integrins, adhesion molecules, glycoproteins, and receptors. Different cell types have different CD antigens. Antibodies recognizing CD antigens are frequently used as an efficient tool in cell sorting and in identifying and characterizing various cell populations. Several CD antigens are associated with mouse and human ESC. The CD antigens associated with pluripotent hES cells are CD9, CD24, and CD133. CD133 is also a hematopoietic stem cell marker. In addition, hES cell express markers such as CD90 and CD117. Another set of markers useful for quantification and characterization of pluripotent stem cells are the integrin set of proteins. These are a/P heterodimeric cell surface receptors that are involved in maintaining the attachment of a cell to its surrounding tissues. They play a pivotal role in cell adhesion, signaling, and migration, as well as in cell growth and survival. Integrins are known to work together with other proteins such as cadherins, immunoglobulin superfamily cell adhesion molecules, selectins, and syndecans, to mediate cell-cell and cell-matrix interaction and communication. They bind to cell surface and ECM components such as fibronectin, vitronectin, collagen, and laminin. Not only integrins perform this outside-in signaling, but they also operate in an inside-out mode. The outside-in signaling via one integrin can promote the activation of another integrin via inside-out signaling. Thus, they transduce information from the ECM to the cell as well as reveal the status of the cell to the outside, allowing rapid and flexible responses to changes in the environment. Multiple types of integrins exist on different cell surfaces, and they play an important role in constructing the environment in which pluripotent stem cells grow. The integrin family contains at least 18 α- and eight β-subunits that form 24 known integrins with distinct tissue distributions and overlapping ligand specificities. The α5β1, αvβ5, α6β1, and α9β1 integrins play important roles in the maintenance of stemness in undifferentiated mouse ESC. Integrin α6 (CD49f/CD29) is a 120-kDa protein with two splice variants, integrins α6A and α6B, which functions as a receptor for laminins and mediates cellular adhesion events on the basal membrane. Integrin α6 (CD49f/CD29) plays an important role in hematopoietic stems and progenitor cells homing to the bone marrow and human prostate carcinoma cells. There are another class of proteins called TRA-1-60 and TRA-1-81 antigens, which originally have been described on the human embryonal carcinoma (EC) cells and human pluripotent stem cell surfaces are widely used as markers in identifying and isolating ESCs. They are also expressed in teratocarcinoma and EG cells. TRA-1-60 antibody reacts with a neuraminidase-sensitive epitope of a proteoglycan, whereas TRA-1-81 reacts with a neuraminidase-insensitive epitope of the same molecule. Recently, this proteoglycan molecule has been proposed as a form of the protein podocalyxin. Another marker associated with pluripotent stem cells is Fzd. This protein is known to be a member of the seven-transmembrane-spanning G-protein-coupled receptor (GPCR) superfamily. Fzd has a large extracellular N-terminal region containing a cysteine-rich domain (CRD), which is involved in the binding to Wnt proteins which are involved in regenerative processes and stem cell renewal. Wnt signals are transduced through the FZD family receptors. The intracellular C-terminus of Fzd binds to the PDZ domain of Dvl proteins, a major signaling component downstream of Fzd. Wnt proteins bind to Fzd and the co-receptors LRP5 or LPR6, and activate the Wnt/β-catenin pathway by inhibiting the phosphorylation of β-catenin by GSK3-β. In addition to this canonical Wnt/β-catenin pathway, some Wnt proteins can also activate the Fzd/Ca2+ and Fzd/PCP (planar cell polarity) pathways. The mammalian Fzd subfamily has 10 members (Fzd1 to Fzd10) and may mediate signaling through different pathways. Some Fzds can also bind to other secreted proteins, such as Norrin and R-Spondin. Fzd 1-10 are expressed in mouse and human pluripotent stem cells.

In one embodiment of the invention, Treg cells are generated from hematopoietic stem cells. Said hematopoietic stem cells are generated from pluripotent stem cells, said hematopoietic stem cells are subsequently treated with GM-CSF and interleukin-4 to generate dendritic cells. In order to generate tolerogenic dendritic cells, treatment of hematopoietic progenitors with interleukin-10 is performed. To generate hematopoietic progenitors numerous means of cellular engineering are known in the art. In one embodiment, Single-cell suspensions of H9 hESCs were obtained by treating hESCs by Gentle Cell Dissociation Reagent (GCDR) (STEMCELL), and then mesodermal EBs were generated on the ultra-low attachment 6-well plates with APEL medium (STEMCELL Technologies) supplemented with 20 ng/mL BMP4, 10 ng/mL Activin A, 25 ng/mL VEGF, 10 ng/mL SCF, 10 ng/mL bFGF (Peprotech), 3 μM CHIR99021, 4 μM SB431542 and with 10 μM Rock inhibitor (Y-27632; STEMCELL Technologies). At day 4, the culture medium was removed and fresh APEL medium containing 20 ng/mL BMP4, 50 ng/mL VEGF, and 10 ng/mL bFGF, 10 ng/mL SCF and 15 ng/mL IGF2 was replenished until day 7. The EBs at day 7 were collected and plated on Matrigel-coated wells to differentiate into HEPs in the presence of APEL differentiation medium I supplemented with 50 ng/mL IL-3, 100 ng/mL SCF, 25 ng/mL Flt3L, 50 ng/mL VEGF, 25 ng/mL IL-6, 25 ng/mL TPO, 3 U/mL EPO, 10 ng/mL bFGF and 20 ng/mL IGF2. From day 11, the last step of the hematopoietic differentiation was carried out in APEL differentiation medium II supplemented with 50 ng/mL VEGF, 100 ng/mL SCF, 25 ng/mL IL-6, 25 ng/mL TPO, 25 ng/mL Flt3L, 10 ng/mL bFGF and 20 ng/mL IGF2 for 4 more days. Cultures were maintained at 37° C. under the normoxia or hypoxia (5% 02) conditions as indicated.

In some embodiments tolerogenic dendritic cells are generated in vivo by administration of MSC that have been pulsed with antigen or tolerogen. Said tolerogenic dendritic cells are utilized to enhance efficacy of administrated Treg. “Mesenchymal stem cell” or “MSC” in some embodiments refers to cells that are (1) adherent to plastic, (2) express CD73, CD90, and CD105 antigens, while being CD14, CD34, CD45, and HLA-DR negative, are of autologous and/or allogeneic origin, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, lxmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

1. A method of generating T regulatory cells comprising the steps of: a) obtaining a pluripotent stem cell population; b) exposing said pluripotent stem cell population to a population of thymic medullary epithelial cells; c) culturing the populations of “a” and “b” together; d) providing conditions for differentiation of said pluripotent stem cells into T regulatory cells and e) further providing conditions for expansion of said T regulatory cells.

2. The method of claim 1, wherein said pluripotent stem cell is an inducible pluripotent stem cell.

3. The method of claim 1, wherein said pluripotent stem cell is substituted with a lymphoid progenitor cell.

4. The method of claim 3, wherein said lymphoid progenitor cell expresses reduced CD127 as compared to T cells.

5. The method of claim 1, wherein said T regulatory cell is capable of suppressing proliferation of peripheral blood mononuclear cells stimulated by ligation of CD3, CD28 and ICAM-1.

6. The method of claim 1, wherein said T regulatory cell is capable of suppressing dendritic cell maturation.

7. The method of claim 6, wherein said T regulatory cell suppresses said dendritic cell maturation in a GITR dependent manner.

8. The method of claim 6, wherein said immature dendritic cell is capable of stimulating generation of anergic T cells in an antigen-dependent manner.

9. The method of claim 6, wherein dendritic cells made immature by Treg cells produce more interleukin-35 as compared to mature dendritic cells.

10. The method of claim 1, wherein said thymic medullary epithelial cells are isolated from living or cadaveric donors.

11. The method of claim 10, wherein said thymic epithelial cells are derived by contacting the cells, or one or more ancestors thereof, with at least one thymic epithelial cell promoting agent, and then allowing or causing the TEPCs to differentiate.

12. The method of claim 11, wherein said promoting agent is mesenchymal stem cell conditioned media.

13. The method of claim 12, wherein said mesenchymal stem cell is perinatal tissue derived.

14. The method of claim 13, wherein said mesenchymal stem cell is activated with TNF-alpha and interferon gamma.

15. The method of claim 1, wherein one or more antigens to which Treg cell generation is desired are placed in the combination of thymic medullary epithelial cells and pluripotent stem cells.

16. The method of claim 15, wherein said antigenic peptides representing said antigen are determined by analysis of peptides capable of binding to HLA-2 alleles representing haplotype of the thymic donor.

17. The method of claim 16, wherein tolerogenic dendritic cells are pulsed with said antigens and administered into a three-dimensional structure containing pluripotent stem cells and thymic medullary epithelial cells.

18. The method of claim 16, wherein said tolerogenic dendritic cells are generated by culture of monocytes in GM-CSF and one or more inhibitors of NF-kappa B.

19. The method of claim 18, wherein said inhibitor of NF-kappa B is minocycline.

20. The method of claim 1, wherein said pluripotent stem cells are combined with said thymic medullary epithelial cells in the presence of a scaffold to facilitate such interaction.
